(19) United States
(12) Patent Application Publication
(10) Pub. No.: US 2017/0105666 A1
(43) Pub. Date: Apr. 20, 2017

(54) COGNITIVE FUNCTION TEST DEVICE AND METHOD

(71) Applicant: Samsung Electronics Co., Ltd., Suwon-si (KR)

(72) Inventor: Ho-Sub LEE, Seoul (KR)

(73) Assignee: Samsung Electronics Co., Ltd., Suwon-si (KR)

(21) Appl. No.: 15/393,798

(22) Filed: Dec. 29, 2016

Related U.S. Application Data
(63) Continuation of application No. PCT/KR2014/006087, filed on Jul. 8, 2014.

(51) Int. Cl. A61B 5/16; G09B 7/06; G09B 7/02; G09B 7/00

(52) U.S. Cl. CPC A61B 5/16; G09B 7/00; G09B 7/06; G09B 7/02

(57) ABSTRACT

Disclosed are cognitive function test device and method, the cognitive function test device including a content information generator configured to generate content information from a content of a user, a question and answer data generator configured to generate at least one question and answer data based on the content information, and a question and answer executor configured to provide a question related to the content and to evaluate an answer to the question based on the at least one question and answer data.

### CROSS-REFERENCE TO RELATED APPLICATION(S)

This application claims the benefit under 35 U.S.C. §§111(a) and 363, and is a continuation of an International Application No. PCT/KR2014/006087 filed on Jul. 8, 2014, the entire disclosure of which is incorporated herein by reference for all purposes.

### BACKGROUND

1. Field

The following description relates to a cognitive function test device and method.

2. Description of Related Art

Dementia is a disease in which multiple cognitive disabilities due to a brain disorder cause difficulty in daily life or social life. This places a great burden, both physically and mentally, on patients and their caregivers. People suffering from dementia are rapidly increasing due to a global aging population situation, but innovative drugs for dementia are not currently available. Thus, it is desirous to diagnose dementia and to receive proper treatment prior to the onset of dementia or in its initial stages.

In a mild cognitive impairment known as a precursor of dementia, abilities related to language, memory, and judgment are degraded, but abilities to perform daily life tasks are often preserved. Since the mild cognitive impairment is an intermediate stage of normal aging and dementia and is a high risk state that may transition to dementia, it is desirous to diagnose the mild cognitive impairment early and to receive proper treatment for preventing dementia and the aggravation of its symptoms.

Basic diagnosis of the mild cognitive impairment is performed through neuropsychological tests and interviews with doctors in most hospitals. These tests and interviews aim to objectively confirm how much a cognitive function of a patient has degraded based on an evaluation standard, which considers age and education. However, since most patients misconstrue the degradation of their cognitive functions as an aging process or determine that their cognitive functions have no problem, they often visit the hospital after the onset of dementia.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In one general aspect, there is provided a cognitive function test device, including a content information generator configured to generate content information from a content of a user, a question and answer data generator configured to generate at least one question and answer data based on the content information, and a question and answer executor configured to provide a question related to the content and to evaluate an answer to the question based on the at least one question and answer data.

The content information may include any one or any combination of a text, a voice, an image, and a video of the content.

The content information may include additional information related to the content.

The at least one question and answer data may include a correct answer for the question.

The question and answer data generator may be configured to determine a priority for each of the at least one question and answer data.

The question and answer executor may be configured to preferentially provide a question included in the at least one question and answer data having a higher priority.

The question and answer data generator may be configured to generate the at least one question and answer data using a question and answer template.

The question and answer executor may be configured to provide a result of the evaluation including any one or any combination of whether the answer is correct and a response time for the question.

The cognitive function test device may include a storage configured to store the at least one question and answer data and a result of the evaluation of the answer.

The question and answer executor may be configured to provide statistical information for evaluation results accumulated during a time period based on the result of the evaluation stored in the storage.

In another general aspect, there is provided a cognitive function test method, including generating content information from a content of a user, generating at least one question and answer data based on the content information, providing a question based on the at least one question and answer data, evaluating an answer for the question, and providing a result of evaluation of the answer.

The content information may include any one or any combination of a text, a voice, an image, and a video of the content.

The content information may include additional information related to the content.

The at least one question and answer data may include a correct answer for the question.

The generating of the at least one question and answer data may include determining a priority for each of the at least one question and answer data.

The providing of the question may include preferentially providing a question included in the at least one question and answer data having a higher priority.

The generating of the at least one question and answer data may include generating the question and answer data using a question and answer template.

The result may include any one or any combination of whether the answer is correct and a response time for the question.

The evaluating of the answer may include storing the result for the answer.

The providing of the result may include providing statistical information for evaluation results accumulated during a time period based on the stored result of the evaluation.

In another general aspect, there is provided a digital device including a touch-sensitive display, a memory configured to store instructions, and a processor configured to execute the instructions to generate content information from a content of a user, to generate at least one question and answer data based on the content information, to output a question related to the content through the touch-sensitive display, and to evaluate an answer to the question based on the at least one question and answer data.

The digital device may include an antenna, and a cellular radio configured to transmit and receive data via the antenna according to a cellular communications standard, wherein the processor is further configured to transmit a result of the evaluation using the cellular radio.

The processor may be configured to generate the content information by extracting a portion of the content, and wherein the portion of the content comprises any one or any combination of a keyword, an important sentence, a character in an image, an image, a series of sequential images from a video, a video segment, and an audio segment.

The content information may include additional information including any one or any combination of a time at which the content is used, a time at which the content is generated, a time at which the content is stored, a title of the content, a type of the content, and a priority of the content.

Other features and aspects will be apparent from the following detailed description, the drawings, and the claims.

### BRIEF DESCRIPTION OF THE DRAWINGS

FIG. 1 is a diagram illustrating an example of a configuration of a cognitive function test device.

FIG. 2 is a diagram illustrating an example of content information.

FIG. 3 is a diagram illustrating an example of a question and answer template.

FIG. 4 is a diagram illustrating an example of question and answer data.

FIGS. 5 to 7 are diagrams illustrating examples of questions provided to a user.

FIG. 8 is a flowchart illustrating a cognitive function test method.

Throughout the drawings and the detailed description, unless otherwise described, the same drawing reference numerals should be understood as referring to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or apparatuses described herein. However, various changes, modifications, and equivalents of the methods, apparatuses, and/or apparatuses described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or apparatuses described herein that will be apparent after an understanding of the disclosure of this application.

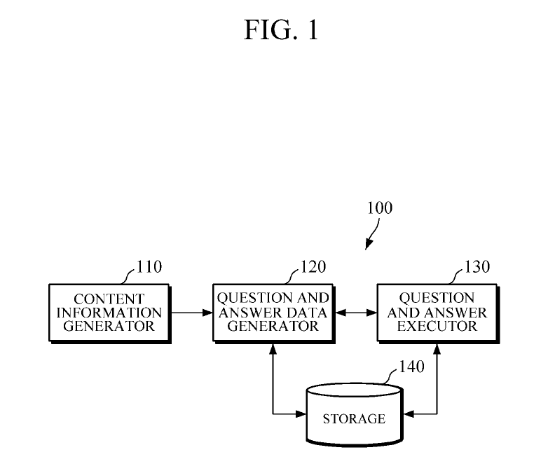

FIG. 1 is a diagram illustrating an example of a configuration of a cognitive function test device through a personalized question and answer. Referring to FIG. 1, a cognitive function test device 100 includes a content information generator 110, a question and answer data generator 120, a question and answer executor 130, and a storage 140.

In an example, the cognitive function test device 100 is included as one component in a terminal capable of providing various types of content such as, for example, text, image, video, or voice. However, the terminal is a non-exhaustive example, and it should be understood that the cognitive function test device 100 may be embedded in or interoperate with various digital devices such as, for example, a mobile phone, a cellular phone, a personal computer (PC), a laptop, a notebook, a subnotebook, a netbook, or an ultra-mobile PC (UMPC), a tablet personal computer (tablet), a phablet, a mobile internet device (MID), a personal digital assistant (PDA), an enterprise digital assistant (EDA), a digital camera, a digital video camera, a portable game console, an MP3 player, a portable/personal multimedia player (PMP), a handheld e-book, an ultra mobile personal computer (UMPC), a portable lab-top PC, a global positioning system (GPS) navigation, a personal navigation device or portable navigation device (PND), a handheld game console, an e-book, and devices such as a high definition television (HDTV), an optical disc player, a DVD player, a Blue-ray player, a setup box, robot cleaners, a home appliance, content players, communication systems, image processing systems, graphics processing systems, other consumer electronics/information technology (CE/IT) device, or any other device capable of wireless communication or network communication consistent with that disclosed herein. In an example, the cognitive function test device 100 may be embedded in or interoperate with a smart appliance, an intelligent vehicle, an electric vehicle, a hybrid vehicle, a smart home environment, or a smart building environment. In an example, the cognitive function test device 100 may also be implemented as a wearable device, which is worn on a body of a user. In one example, a wearable device may be self-mountable on the body of the user, such as, for example, a watch, or an eye glass display (EGD), which includes one-eyed glass or two-eyed glasses.

In an example, the content information generator 110 generates content information related to content used by the user.

In an example, the content information generator 110 collects various types of content that the user produces and consumes through various cognitive activities, such as, for example, reading, writing, speaking, hearing, when using the terminal. The content information generator 110 may generate content information from the collected content. In an example, the content may include at least one of text, a voice, an image, and a video, and the content information may include at least one portion of the text, voice, image, or video included in the collected content.

For example, the content information may include an important sentence or a keyword extracted from the text included in the content. When the content includes text, the content information generator 110 may extract an important sentence or keyword from the text included in the content. In an example, natural language processing technology or document summarizing technology may be used for extracting the important sentence or keyword. However, the methods for extraction are only non-exhaustive examples, and other methods, such as, for example, extracting text of a portion that the user actually reads in the content using eye-tracking by a camera included in the terminal are considered to be well within the scope of the present disclosure.

As another example, the content information may include an important sentence or keyword extracted from the voice included in the content. When the collected content includes the voice, the content information generator 110 may recognize the voice included in the content, convert the recognized voice into text, and extract an important sentence or keyword from the converted text. In an example, the content information generator 110 may recognize and convert the voice included in the content into text using speech-to-text technology such as, for example, hidden Markov model (HMM), dynamic time warping (DTW), or neural network.

In another example, when the collected content includes video, the content information may include a portion of video extracted from the video included in the content or an image captured from the video.

In another example, when the collected content includes an image, the content information may include the image included in the content.

In an example, the content information includes additional information regarding the collected content, such as, for example, a time at which the content is used by the user, a time at which the content is generated or stored, or a type of the content. The examples of additional information mentioned above are only non-exhaustive illustrations, and other types of additional information collected according to a type or shape of the content are considered to be well within the scope of the present disclosure.

For example, when the content is a video, the additional information includes information such as, for example, a title of the video, a character in the video, and a time at which the video is played. As another example, when the content is music, the additional information includes information such as, for example, a title, a singer, a genre, a player, and a time at which the music is played.

As another example, when the content is a text message or e-mail, the additional information includes information such as, for example, a time at which the text message or e-mail is received or transmitted and a recipient or sender of the text message or e-mail, a subject of the text message or e-mail, and a priority of the text message or e-mail.

As another example, when the content is voice communication, the additional information includes information such as, for example, a voice communication time and a communication counterpart.

In an example, the question and answer data generator 120 generates at least one piece of question and answer data based on the content information generated by the content information generator 110. In an example, the question and answer data includes a question related to the content used by the user and a correct answer for the question.

For example, the question and answer data may include a question related to at least one among the important sentence and the keyword included in the content information, the image, the video, and the voice and, and a correct answer for the question.

As another example, the question and answer data may include a question related to the additional information included in the content information, and a correct answer for the question.

In an example, the question and answer data generator 120 generates at least one piece of question and answer data using a question and answer template that is previously defined. The question and answer data generator 120 generates at least one piece of question and answer data by applying the content information generated by the content information generator 110 to the question and answer template. In an example, the question and answer template may have various forms such as, for example, one in which the keyword is inserted into the important sentence, one in which a time is guessed using the content, and one in which the type of the content in which the important sentence is included is guessed. In an example, the question and answer template may be added to or corrected by the user.

In an example, the question and answer data generator 120 determines a priority for the generated question and answer data. In an example, a criterion of determining the priority is previously defined, and is set or changed by the user.

For example, the question and answer data generator 120 assigns the question and answer data to have a lower priority than the question and answer data related to content that is recently used based on a recoding time of the content.

As another example, the question and answer data generator 120 assigns a higher priority to the question and answer data than the question and answer data related to a type of content having the smallest frequency (for example, an e-mail) of use by the user.

As another example, the question and answer data generator 120 assigns the question and answer data to have a lower priority than the question and answer data that is most recently performed based on a time at which a question and answer is performed by the user.

As another example, the question and answer data generator 120 assigns the question and answer data to have a higher priority as a length of an important sentence included in the question and answer data increases or the number of keywords included in the important sentence increases.

The criteria for determining the priority for the question and answer data is not limited to the examples described above, and the priority may be determined according to various other standards, without departing from the spirit and scope of the illustrative examples described. For example, the priority for the question and answer data may be updated, deleted, or changed according to criteria such as, for example, a lapse of time, the number of questions, and an addition of new question and answer data.

In an example, the question and answer executor 130 provides a question related to the content used by the user based on the question and answer data generated by the question and answer data generator 120. In an example, the question and answer executor 130 evaluates an answer of the user for the provided question.

In an example, the question and answer executor 130 provides a question included in the question and answer data to the user according to a predetermined period of time or when a specific event is generated. For example, the question and answer executor 130 periodically evaluates an answer by providing the question included in the question and answer data to the user according to the predetermined period and obtaining an answer. In an example, the period may be set or changed by the user.

As another example, when specific content is used by the user, the question and answer executor 130 provides a question included in the question and answer data related to the corresponding content. When the user plays video content, the question and answer executor 130 may provide a question included in the question and answer data related to the video content to the user.

In an example, the question and answer executor 130 preferentially provides a question having a higher priority among the question and answer data to the user based on the priority determined by the question and answer data generator 120.

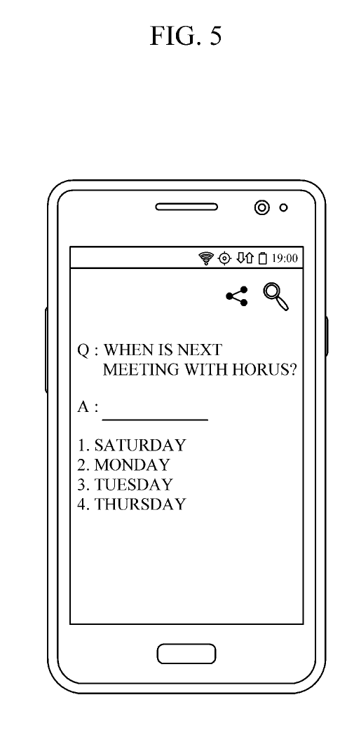

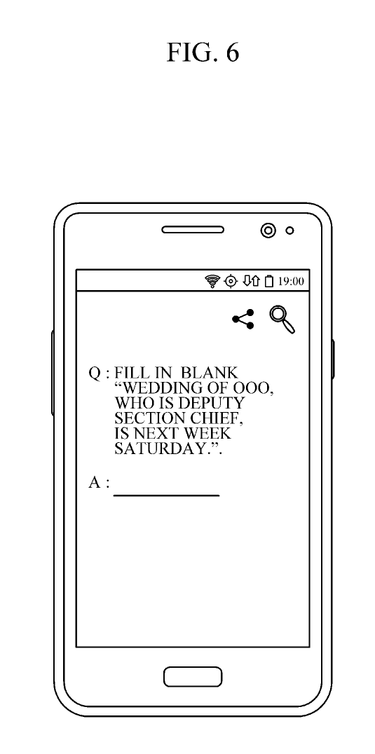

In an example, the question and answer executor 130 provides a question included in the question and answer data to the user in the form of text or voice. For example, as shown in FIGS. 5 and 6, the question and answer executor 130 displays the question included in the question and answer data on a screen of the terminal in the form of text.

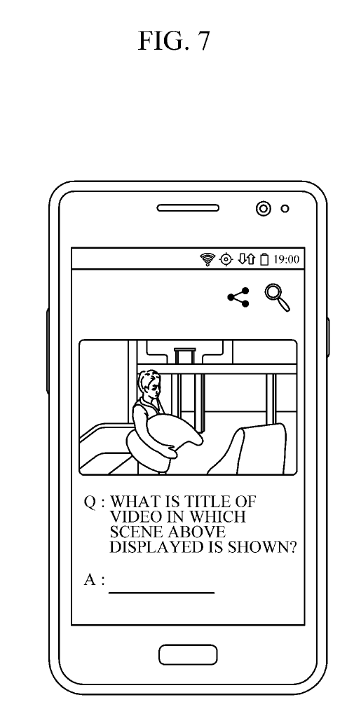

In another example, as shown in FIG. 7, the question and answer executor 130 displays the question included in the question and answer data on the screen of the terminal as a combination of an image and text.

The form of the question provided to the user is not limited to the examples shown in FIGS. 5 to 7, various others forms may be used without departing from the spirit and scope of the illustrative examples described. For example, the question provided to the user may be provided in a combination of one or more of a voice, text, image, and video.

The user may provide an answer to the provided question in various manners using an input means included in the terminal. For example, the user may input the answer using a keypad or a touch screen included in the terminal. As another example, the user may input the answer in the form of voice through a microphone included in the terminal.

When the user's answer is input, the question and answer executor 130 evaluated the answer input by the user, and provide an evaluation result to the user. For example, the question and answer executor 130 may compare the answer of the user and the correct answer included in the question and answer data, determine whether the answer of the user is correct, and provide the determined result to the user. In another example, the question and answer executor 130 may measure a response time of the user for the provided question, and provide the measured response time to the user.

In an example, the question and answer executor 130 provides statistical information such as, for example, evaluation results accumulated during a period of time to based on evaluation results for the question and answer data stored in the storage 140. In an example, the accumulated evaluation results may include a correct answer rate for the question and answer performed during the period of time or statistical information on an average response time. In an example, the period of time is predetermined.

In an example, the question and answer executor 130 provides a mild cognitive impairment diagnosis advisory message to the user based on the accumulated evaluation results. For example, the question and answer executor 130 provides the mild cognitive impairment diagnosis advisory message to the user when the evaluation result continuously degrades, such that the user may not miss a treatment period. In an example, the evaluation result degrades when the correct answer rate continuously decreases, the correct answer rate is of a predetermined level or less, or the response time continuously increases.

The storage 140 may store the question and answer data and the evaluation result for the question and answer data in a non-transitory computer-readable storage medium. Various non-exhaustive example of the storage 140 has been provided below.

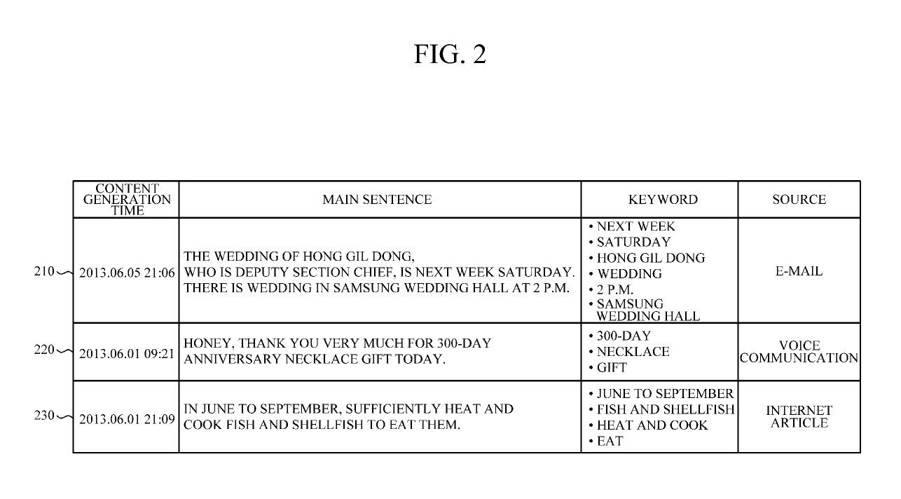

FIG. 2 is a diagram illustrating an example of content information.

The content information 210, 220, and 230 of the example shown in FIG. 2 includes an important sentence, keywords extracted from the content, and may include the content from which an important sentence and keyword are extracted. The content information may also include a generation time of corresponding content as additional information. The content information shown in FIG. 2 is merely an example for explanation, and other types of content information are considered to be well within the scope of the present disclosure.

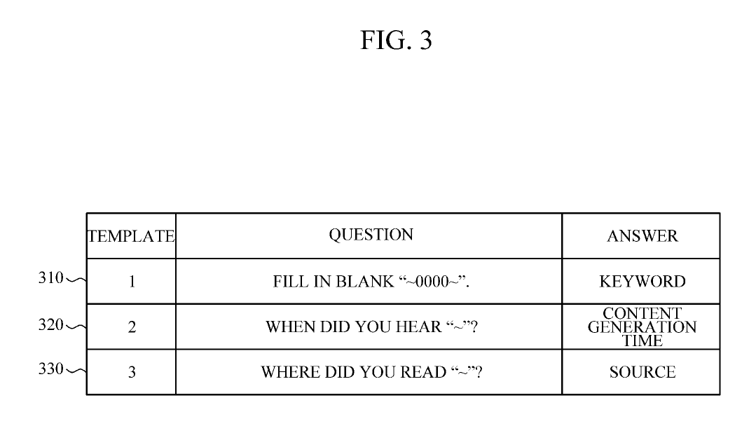

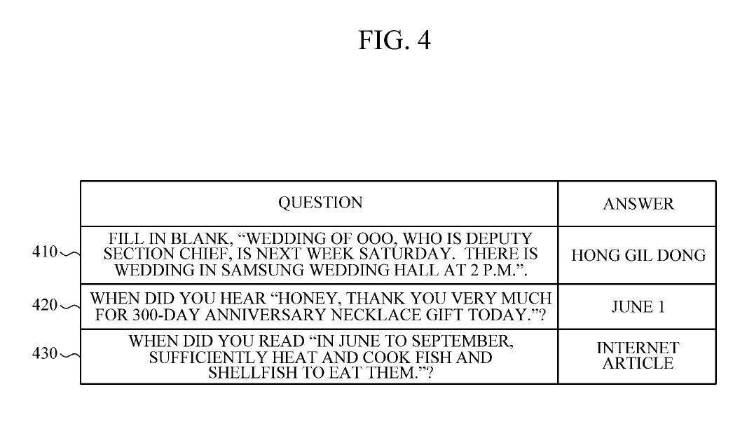

FIG. 3 is a diagram illustrating an example of a question and answer template, and FIG. 4 is a diagram illustrating an example of question and answer data.

In an example, in FIG. 3, a template 310 may be for generating a question having a form in which a keyword is inserted into an important sentence included in content information. For example, the question and answer data generator 120 may apply content information 210 shown in FIG. 2 to the template 310, and generate the question and answer data shown in 410 of FIG. 4.

In another example, in FIG. 3, a template 320 may be for generating a question having a form that asks for a time at which an important sentence included in content information is heard. For example, the question and answer data generator 120 may apply content information 220 shown in FIG. 2 to the template 320, and generate the question and answer data shown in 420 of FIG. 4.

In another example, in FIG. 3, a template 330 may be for generating a question having a form that asks for a source of an important sentence included in content information. For example, the question and answer data generator 120 may apply content information 230 shown in FIG. 2 to the template 330, and generate the question and answer data shown in 430 of FIG. 4.

Meanwhile, the examples shown in FIGS. 3 and 4 are merely examples for explanation, and other types of question and answer template and question and answer data are considered to be well within the scope of the present disclosure.

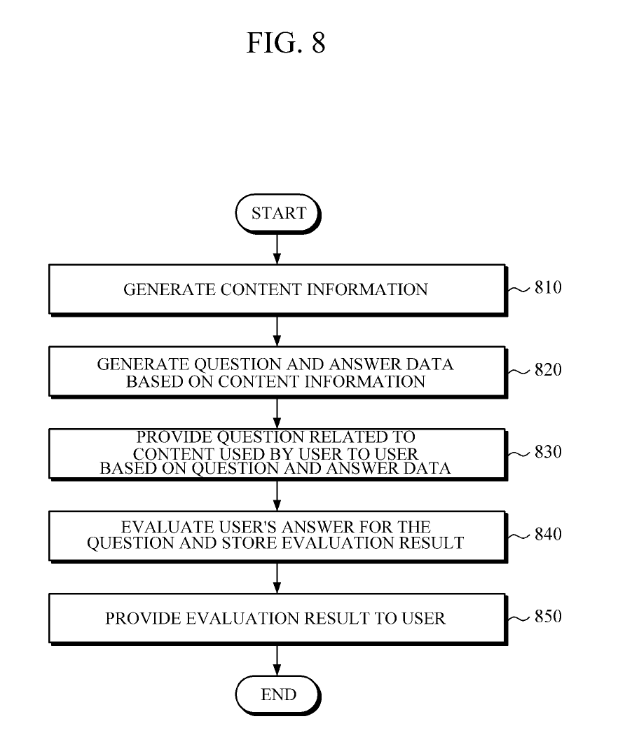

FIG. 8 is a diagram illustrating an example of a cognitive function test method. The operations in FIG. 8 may be performed in the sequence and manner as shown, although the order of some operations may be changed or some of the operations omitted without departing from the spirit and scope of the illustrative examples described. Many of the operations shown in FIG. 8 may be performed in parallel or concurrently. In addition to the description of FIG. 8 below, the above descriptions of FIGS. 1-7, are also applicable to FIG. 8, and are incorporated herein by reference. Thus, the above description may not be repeated here.

Referring to FIG. 8, in 810, the cognitive function test device 100 generates content information related to content used by a user. In an example, the content information includes at least one portion among text, a voice, an image, or a video included in the content, or additional information related to the content.

In 820, the cognitive function test device 100 generates at least one piece of question and answer data based on the generated content information. In an example, the question and answer data includes a question related to the content used by the user and a correct answer for a corresponding question.

In an example, the cognitive function test device 100 generates the question and answer data by applying the generated content information to at least one question and answer template.

In an example, the cognitive function test device 100 determines a priority for the generated question and answer data.

In 830, the cognitive function test device 100 provides the question related to the content used by the user to the user based on the question and answer data.

In an example, the cognitive function test device 100 provides the question included in the question and answer data to the user according to a predetermined period or whether a specific event is generated.

In an example, the cognitive function test device 100 preferentially provides a question included in the question and answer data having a higher priority among the question and answer data.

In 840, the cognitive function test device 100 evaluates an answer of the user for the provided question, and store a result of the evaluation. In an example, the evaluation result may include whether the answer of the user is correct or a response time of the user.

In 850, the cognitive function test device 100 provides the evaluation result for the provided question to the user (S850). In an example, the cognitive function test device 100 provides statistical information on evaluated results accumulated during a predetermined period to the user based on the stored evaluated result. For example, the accumulated evaluated results may include a correct answer rate for question and answer performed during the predetermined period or statistical information on an average response time.

Since a question and answer related to content used by a user is performed, it is possible to perform a user personalized cognitive function test and long-term memory as well as short-term memory can be evaluated. Progression of the mild cognitive impairment to dementia can be prevented, since the chance of the user missing a treatment for the mild cognitive impairment is reduced by providing an evaluation result for the question and answer to the user. Since the evaluation result for the question and answer of the user is stored, medical staff are able to use the stored evaluation result to diagnose and treat the mild cognitive impairment.

The cognitive function test device 100, content information generator 110, question and answer data generator 120, question and answer executor 130 described in FIG. 1 that perform the operations described in this application are implemented by hardware components configured to perform the operations described in this application that are performed by the hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIG. 8 that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A cognitive function test device, comprising:
a content information generator configured to generate content information from a content of a user;
a question and answer data generator configured to generate at least one question and answer data based on the content information; and
a question and answer executor configured to provide a question related to the content and to evaluate an answer to the question based on the at least one question and answer data.

2. The cognitive function test device of claim 1, wherein the content information comprises any one or any combination of a text, a voice, an image, and a video of the content.

3. The cognitive function test device of claim 1, wherein the content information comprises additional information related to the content.

4. The cognitive function test device of claim 1, wherein the at least one question and answer data comprises a correct answer for the question.

5. The cognitive function test device of claim 1, wherein the question and answer data generator is further configured to determine a priority for each of the at least one question and answer data.

6. The cognitive function test device of claim 5, wherein the question and answer executor is further configured to preferentially provide a question included in the at least one question and answer data having a higher priority.

7. The cognitive function test device of claim 1, wherein the question and answer data generator is further configured to generate the at least one question and answer data using a question and answer template.

8. The cognitive function test device of claim 1, wherein the question and answer executor is further configured to provide a result of the evaluation comprising any one or any combination of whether the answer is correct and a response time for the question.

9. The cognitive function test device of claim 1, further comprising:
a storage configured to store the at least one question and answer data and a result of the evaluation of the answer.

10. The cognitive function test device of claim 9, wherein the question and answer executor is further configured to provide statistical information for evaluation results accumulated during a time period based on the result of the evaluation stored in the storage.

11. A cognitive function test method, comprising:
generating content information from a content of a user;
generating at least one question and answer data based on the content information;
providing a question based on the at least one question and answer data;
evaluating an answer for the question; and
providing a result of evaluation of the answer.

12. The cognitive function test method of claim 11, wherein the content information comprises any one or any combination of a text, a voice, an image, and a video of the content.

13. The cognitive function test method of claim 11, wherein the content information comprises additional information related to the content.

14. The cognitive function test method of claim 11, wherein the at least one question and answer data comprises a correct answer for the question.

15. The cognitive function test method of claim 11, wherein the generating of the at least one question and answer data comprises determining a priority for each of the at least one question and answer data.

16. The cognitive function test method of claim 15, wherein the providing of the question comprises preferentially providing a question included in the at least one question and answer data having a higher priority.

17. The cognitive function test method of claim 11, wherein the generating of the at least one question and answer data comprises generating the question and answer data using a question and answer template.

18. The cognitive function test method of claim 11, wherein the result comprises any one or any combination of whether the answer is correct and a response time for the question.

19. The cognitive function test method of claim 11, wherein the evaluating of the answer comprises storing the result for the answer.

20. The cognitive function test method of claim 19, wherein the providing of the result comprises providing statistical information for evaluation results accumulated during a time period based on the stored result of the evaluation.

21. A non-transitory computer-readable storage medium storing instructions that, when executed by a processor, causes the processor to perform the method of claim 11.

22. A digital device comprising:
a touch-sensitive display;
a memory configured to store instructions; and
a processor configured to execute the instructions to generate content information from a content of a user, to generate at least one question and answer data based on the content information, to output a question related to the content through the touch-sensitive display, and to evaluate an answer to the question based on the at least one question and answer data.

23. The digital device of claim 22, further comprising:
an antenna; and
a cellular radio configured to transmit and receive data via the antenna according to a cellular communications standard,
wherein the processor is further configured to transmit a result of the evaluation using the cellular radio.

24. The digital device of claim 22, wherein the processor is further configured to generate the content information by extracting a portion of the content, and wherein the portion of the content comprises any one or any combination of a keyword, an important sentence, a character in an image, an image, a series of sequential images from a video, a video segment, and an audio segment.

25. The digital device of claim 22, wherein the content information further comprises additional information comprising any one or any combination of a time at which the content is used, a time at which the content is generated, a time at which the content is stored, a title of the content, a type of the content, and a priority of the content.
